(19) United States
(12) Patent Application Publication
(10) Pub. No.: US 2024/0017020 A1
(43) Pub. Date: Jan. 18, 2024

(54) MECHANISM FOR RECEIVING AN INJECTOR, AND MEDICAMENT DELIVERY DEVICE

(71) Applicant: SHL MEDICAL AG, ZUG (CH)

(72) Inventor: Daniel Säll, Segeltorp (SE)

(21) Appl. No.: 18/035,996

(22) PCT Filed: Nov. 19, 2021
(86) PCT No.: PCT/EP2021/082272

§ 371 Date: May 9, 2023

(30) Foreign Application Priority Data
Dec. 3, 2020 (EP) 20211641.4

(51) Int. Cl. A61M 5/32

(52) U.S. Cl. CPC A61M 5/3287; A61M 5/3204

(57) ABSTRACT

A mechanism for receiving an injector having a needle and a compressible body for driving expulsion of medicament through the needle, the mechanism having an actuating element configured to transition from a ready state to a dosing state by means of manual manipulation; a pressing device arranged to move from a starting position to a pressing position to thereby press the compressible body when the injector is received by the mechanism; and a force transmission arrangement configured to transmit a transition of the actuating element from the ready state to the dosing state to a movement of the pressing device from the starting position to the pressing position. A medicament delivery device comprising the injector and a mechanism is also provided.

### TECHNICAL FIELD

The present disclosure generally relates to a mechanism for receiving an injector. In particular, a mechanism for receiving an injector having a needle and a compressible body for driving expulsion of medicament through the needle, and a medicament delivery device comprising the injector and such mechanism, are provided.

### BACKGROUND

Blow-Fill-Seal (BFS) technology is a manufacturing technique used to produce containers containing a medicament, such as a liquid. BFS syringes can be manufactured in very high volumes in relatively short time. One application for such BFS syringes is to provide vaccine against COVID-19. A BFS syringe may be provided in a package, such as a foil package. An accurate single dose of medicament can thereby be provided by one such package.

However, many types of BFS syringes are not suitable for home use. For example, many BFS syringes lack safety equipment, such as needle protection after use.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

One object of the present disclosure is to provide a mechanism for receiving an injector having a needle and a compressible body for driving expulsion of medicament through the needle, which mechanism enables a simple use.

A further object of the present disclosure is to provide a mechanism for receiving an injector having a needle and a compressible body for driving expulsion of medicament through the needle, which mechanism enables a safe use.

A still further object of the present disclosure is to provide a mechanism for receiving an injector having a needle and a compressible body for driving expulsion of medicament through the needle, which mechanism enables a reliable use.

A still further object of the present disclosure is to provide a mechanism for receiving an injector having a needle and a compressible body for driving expulsion of medicament through the needle, which mechanism solves several or all of the foregoing objects in combination.

A still further object of the present disclosure is to provide a medicament delivery device comprising the injector and a mechanism, which medicament delivery device solves one, several or all of the foregoing objects.

According to one aspect, there is provided a mechanism for receiving an injector having a needle and a compressible body for driving expulsion of medicament through the needle, the mechanism comprising an actuating element configured to transition from a ready state to a dosing state by means of manual manipulation; a pressing device arranged to move from a starting position to a pressing position to thereby press the compressible body when the injector is received by the mechanism; and a force transmission arrangement configured to transmit a transition of the actuating element from the ready state to the dosing state to a movement of the pressing device from the starting position to the pressing position.

By means of manual manipulation of the actuating element, the mechanism enables medicament to be reliably expelled through the needle of the injector, e.g. into an injection site, in a simple manner. The actuating element may for example be manually pushed to cause the transition of the actuating element from the ready state to the dosing state. The push may be made by a finger of a user. Alternatively, the push may be provided by gripping the mechanism, contacting an injection site by means of the actuating element, and pushing the mechanism towards the injection site.

The mechanism may comprise a longitudinal axis. The needle may be concentric with the longitudinal axis when the injector is received by the mechanism.

The actuating element may be substantially centered, or centered, with respect to the longitudinal axis. Alternatively, or in addition, the pressing device may be substantially centered, or centered, with respect to the longitudinal axis.

The pressing device may provide a substantially radially inwardly directed force, with respect to the longitudinal axis, against the compressible body to compress the compressible body. The compressible body may be a bubble.

The actuating element may be movable from the ready state to the dosing state. In this case, the actuating element may be rigid, and the ready state and the dosing state may be a ready position and a dosing position, respectively. Alternatively, the actuating element may be elastic. In this case, the actuating element may elastically deform from the ready state to the dosing state.

The mechanism may further comprise a mechanism body. The mechanism body may for example be a housing. The actuating element may move relative to the mechanism body in order to transition from the ready state to the dosing state. The pressing device may move relative to the body.

The injector may be a single-dose injector. Alternatively, or in addition, the injector may be a syringe, such as a Blow-Fill-Seal (BFS) syringe.

The force transmission arrangement may comprise a cam profile and a cam follower arranged to follow the cam profile. The cam follower may for example be a pin. The force transmission arrangement according to the present disclosure may however be realized in alternative ways, for example by means of one or more linkages.

The force transmission arrangement may comprise a movable member. In this case, the cam profile or the cam follower may be provided on the movable member.

The cam profile or the cam follower may be provided on the pressing device. Thus, the cam profile may be provided on the movable member and the cam follower may be provided on the pressing device, or vice versa.

The mechanism may further comprise a needle cover. The needle cover may be configured to transition from an exposed state, where the needle cover does not cover the needle, to a covered state, where the needle cover covers the needle. The mechanism may further comprise a cover force device arranged to force the needle cover towards the covered state. The needle cover thus ensures that the user is protected from needle sticks after completion of medicament delivery. The cover force device may be a spring, such as a compression coil spring. The cover force device may be arranged between the mechanism body and the needle cover.

The needle cover may be arranged at a proximal end of the mechanism. The needle cover may be substantially centered, or centered, with respect to the longitudinal axis.

The needle cover may be prevented by the pressing device from transitioning to the covered state when the pressing device adopts the starting position, and allowed by the pressing device to transition to the covered state when the pressing device adopts the pressing position. In this way, it can be ensured that the needle cover transitions to the covered state only when the medicament delivery has been completed.

The actuating element may comprise the needle cover. Thus, the actuating element may be arranged at a proximal end of the mechanism.

Alternatively, the actuating element may comprise a button. The button may be arranged at a distal end of the mechanism.

The pressing device may comprise two arms. The two arms may be arranged to move towards each other when the pressing device moves from the starting position to the pressing position. Each arm may be rotatable towards each other. To this end, the pressing device may further comprise two hinges. Each arm may thus be rotatable about a respective hinge. Each hinge may be a living hinge. Alternatively, or in addition, each hinge may be substantially perpendicular to, or perpendicular to, the longitudinal axis.

The mechanism may further comprise a locking arrangement. The locking arrangement may be arranged to lock the needle cover in the covered state after having transitioned from the exposed state to the covered state. By means of the locking arrangement, it can be ensured that the needle is not accidentally exposed after completion of the medicament delivery.

The injector may comprise a tab on a distal side of the compressible body. In this case, the mechanism may comprise a gripping structure arranged to grip the tab when the injector is received by the mechanism. The gripping structure enables the injector to be inserted into the mechanism and to be reliably held by the mechanism. The gripping structure may comprise one or more gripping fingers or gripping claws, for example made of metal.

The gripping structure may be provided in the pressing device.

The injector may further comprise a needle shield arranged to be unscrewed to expose the needle. In this case, the mechanism may further comprise a cap arranged to rotationally engage the needle shield by rotation of the cap. In this way, the needle shield is better protected and accidental removal of the needle shield can be avoided. The needle shield may be a rigid needle shield (RNS).

The cap may be configured such that the cap rotationally engages the needle shield by rotation in a first direction, and such that the cap does not rotationally engage the needle shield by rotation in a second direction, opposite to the first direction. In this way, correct removal of the cap can be ensured and avoidance of damage of the injector can be avoided further.

The cap may be provided with an insert, for example made of metal. By means of the insert, the cap and the needle shield can provide a slip/grip device or a freewheel device.

The mechanism may further comprise a pressing force device arranged to force the pressing device to the pressing position. The pressing force device may be arranged between the mechanism body and the movable member.

The mechanism may be configured such that a force from the pressing force device is released when the actuating element adopts the dosing state. For example, the actuating element may comprise a leg and the movable member may comprise an engageable structure. When the actuating element adopts the ready state, the leg may block the engageable structure such that the movable member is prevented from moving in a proximal direction. When the actuating element moves from the ready state to the dosing state, the leg may move away from the position blocking the proximal protrusion such that the movable member is allowed to move in a proximal direction.

The pressing force device may comprise a spring. The spring may be a compression coil spring.

The pressing force device may be arranged to force the movable member. The pressing force device may force the movable member in the proximal direction.

The actuating element may be configured to prevent movement of the pressing device to the pressing position when the actuating element adopts the ready state.

The mechanism may comprise a mechanism body having at least one window through which a state of the mechanism and/or the injector indicative of medicament having been expelled through the needle is visible. For example, the mechanism as such may be visible through one window and/or the compressible body may be visible through one window. The at least one window may comprise a distal window through which the pressing device is visible and a proximal window through which a medicament container of the injector is visible.

According to a further aspect, there is provided a medicament delivery device comprising the injector and a mechanism according to the present disclosure. The injector may be of any type as described herein. Each of the mechanism and the injector may be disposable.

The insertion of the injector to the mechanism may be made at a point-of-care. In this case, the user may obtain the injector and the mechanism through different channels.

Alternatively, the injector may be inserted into the mechanism at a factory. In this case, the assembled medicament delivery device may be shipped in a single package.

### BRIEF DESCRIPTION OF THE DRAWINGS

Further details, advantages and aspects of the present disclosure will become apparent from the following description taken in conjunction with the drawings, wherein:

FIG. 1: schematically represents a perspective and partially exploded view of a medicament delivery device;

FIG. 2: schematically represents a perspective view of the medicament delivery device;

FIG. 3A: schematically represents a first cross-sectional side view of the medicament delivery device;

FIG. 3B: schematically represents a second cross-sectional side view of the medicament delivery device in FIG. 3A;

FIG. 4A: schematically represents a first cross-sectional side view of the medicament delivery device after removal of a needle shield;

FIG. 4B: schematically represents a second cross-sectional side view of the medicament delivery device in FIG. 4A;

FIG. 5A: schematically represents a first cross-sectional side view of the medicament delivery device when an actuating element has transitioned to a dosing state;

FIG. 5B: schematically represents a second cross-sectional side view of the medicament delivery device in FIG. 5A;

FIG. 5C: schematically represents a partial perspective view of the medicament delivery device in FIGS. 5A and 5B;

FIG. 6A: schematically represents a partial perspective view of the medicament delivery device when a pressing device has moved to a pressing position;

FIG. 6B: schematically represents a first cross-sectional side view of the medicament delivery device in FIG. 6A;

FIG. 6C: schematically represents a second cross-sectional side view of the medicament delivery device in FIGS. 6A and 6B;

FIG. 7A: schematically represents a first cross-sectional side view of the medicament delivery device when the actuating element has transitioned to a ready state;

FIG. 7B: schematically represents a second cross-sectional side view of the medicament delivery device in FIG. 7A;

FIG. 8: schematically represents a perspective and partially exploded view of a medicament delivery device according to a further example;

FIG. 9A: schematically represents a first cross-sectional side view of the medicament delivery device in FIG. 8;

FIG. 9B: schematically represents a second cross-sectional side view of the medicament delivery device in FIG. 9A;

FIG. 10A: schematically represents a first cross-sectional side view of the medicament delivery device in FIGS. 8 to 9B after removal of a needle shield;

FIG. 10B: schematically represents a second cross-sectional side view of the medicament delivery device in FIG. 10A;

FIG. 10C: schematically represents a first partial perspective view of the medicament delivery device in FIGS. 10A and 10B;

FIG. 10D: schematically represents a second partial perspective view of the medicament delivery device in FIGS. 10A and 10B;

FIG. 11A: schematically represents a first partial perspective view of the medicament delivery device in FIGS. 8 to 10D when an actuating element has been moved to a dosing state;

FIG. 11B: schematically represents a second partial perspective view of the medicament delivery device in FIG. 11A;

FIG. 11C: schematically represents a first cross-sectional side view of the medicament delivery device in FIGS. 11A and 11B;

FIG. 11D: schematically represents a second cross-sectional side view of the medicament delivery device in FIGS. 11A and 11B;

FIG. 12A: schematically represents a first cross-sectional side view of the medicament delivery device in FIGS. 8 to 11C during proximal movement of a needle cover;

FIG. 12B: schematically represents a second cross-sectional side view of the medicament delivery device in FIG. 12A;

FIG. 13A: schematically represents a first cross-sectional side view of the medicament delivery device in FIGS. 8 to 12B when the needle cover has transitioned to a covered state;

FIG. 13B: schematically represents a second cross-sectional side view of the medicament delivery device in FIG. 13A;

FIG. 14: schematically represents a perspective and partially exploded view of a medicament delivery device according to a further example;

FIG. 15A: schematically represents a first cross-sectional side view of the medicament delivery device in FIG. 14;

FIG. 15B: schematically represents a second cross-sectional side view of the medicament delivery device in FIG. 15A;

FIG. 15C: schematically represents a partial perspective view of the medicament delivery device in FIGS. 15A and 15B;

FIG. 16A: schematically represents a partial perspective view of the medicament delivery device in FIGS. 14 to 15C when a pressing device has moved to a pressing position;

FIG. 16B: schematically represents a first cross-sectional side view of the medicament delivery device in FIG. 16A; and

FIG. 16C: schematically represents a second cross-sectional side view of the medicament delivery device in FIGS. 16A and 16B.

### DETAILED DESCRIPTION

In the following, a mechanism for receiving an injector having a needle and a compressible body for driving expulsion of medicament through the needle, and a medicament delivery device comprising the injector and such mechanism, will be described. The same or similar reference numerals will be used to denote the same or similar structural features.

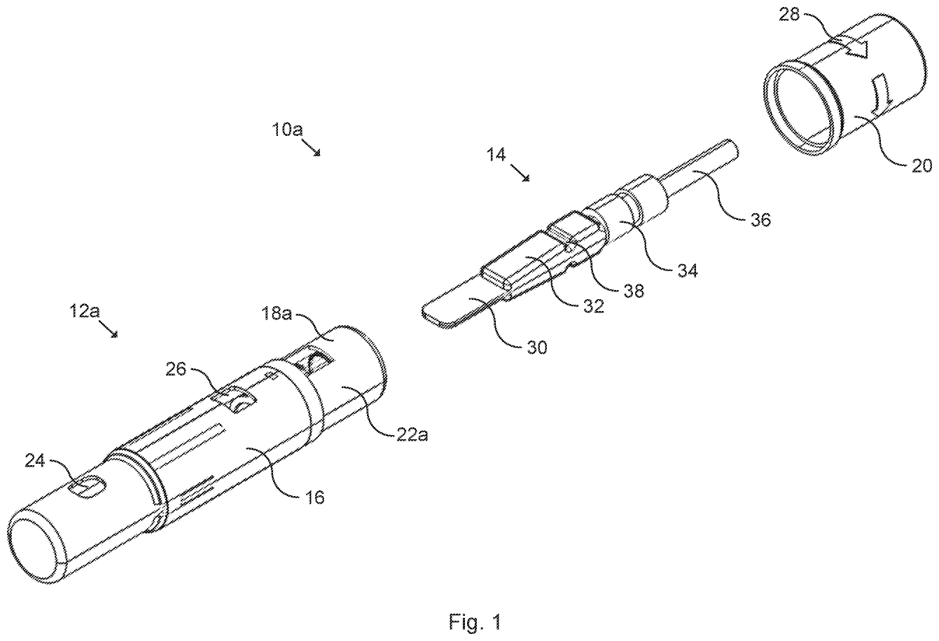

FIG. 1 schematically represents a perspective and partially exploded view of the medicament delivery device 10a. The medicament delivery device 10a comprises a mechanism 12a and a syringe 14.

The mechanism 12a comprises a housing 16, a needle cover 18a and a cap 20. In this example, the needle cover 18a constitutes one example of an actuating element 22a according to the present disclosure. The needle cover 18a is provided at a proximal end of the mechanism 12a.

The housing 16 is one example of a mechanism body according to the present disclosure. The housing 16 comprises a distal window 24 and a proximal window 26.

The cap 20 is rotatable in a first direction 28. In this example, the first direction 28 is indicated with arrows on the cap 20.

The syringe 14 is one example of an injector according to the present disclosure. The syringe 14 of this example is a single-dose BFS syringe. The syringe 14 comprises a tab 30, a bubble 32, a needle hub 34, a needle (not visible) and a needle shield 36. The bubble 32 is one example of a compressible body according to the present disclosure. The bubble 32 is provided with two opposite recesses 38. The syringe 14, or at least the bubble 32, may be made of a transparent material, such as plastic.

The syringe 14 further comprises a medicament reservoir (not denoted). The needle can penetrate into the medicament reservoir by twisting or pushing the needle shield 36. The needle shield 36 is here a rigid needle shield (RNS). The medicament can be expelled through the needle for injection by pressing the bubble 32.

The tab 30 may be provided with various information associated with the syringe 14, such as information regarding the type and/or volume of the medicament. The information may be provided as written information and/or in an RFID (radio-frequency identification) tag.

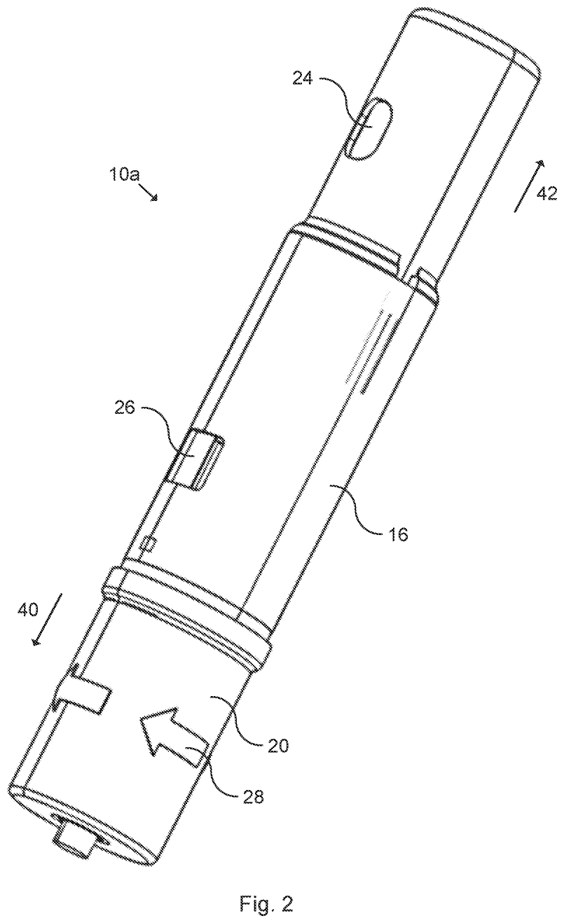

FIG. 2 schematically represents a perspective view of the medicament delivery device 10a. In FIG. 2, the medicament delivery device 10a is assembled such that the syringe 14 is held by the mechanism 12a and the cap 20 is mounted over the needle shield 36. The medicament delivery device 10a may be delivered pre-assembled with the cap 20, i.e. according to the state in FIG. 2.

FIG. 2 further shows a proximal direction 40 and a distal direction 42, opposite to the proximal direction 40, of the medicament delivery device 10a. The cap 20 is provided at a proximal end of the medicament delivery device 10a.

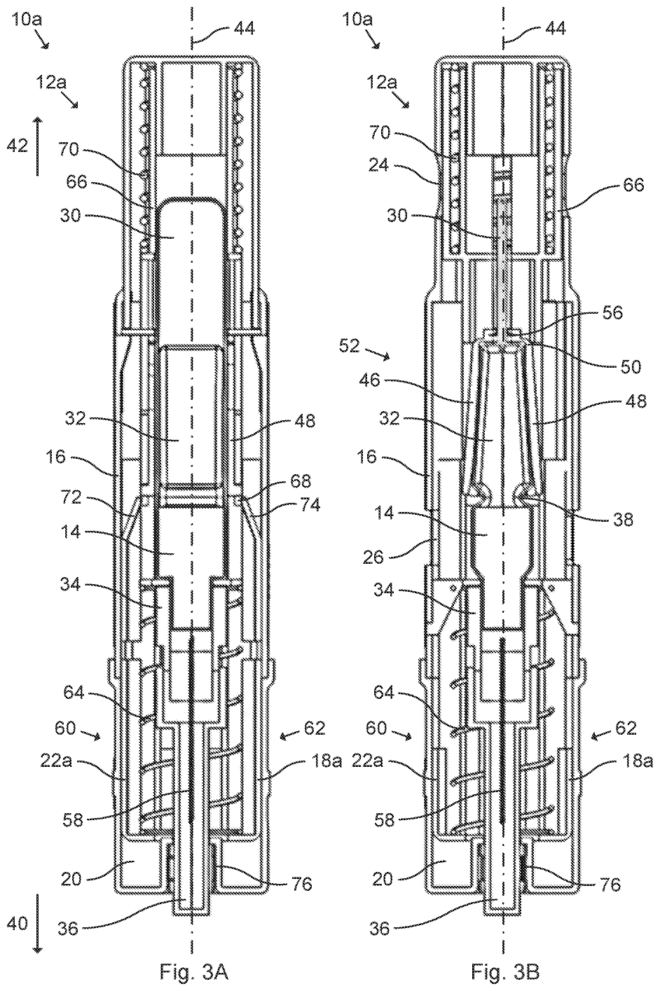

FIG. 3A schematically represents a first cross-sectional side view of the medicament delivery device 10a, and FIG. 3B schematically represents a second cross-sectional side view of the medicament delivery device 10a in FIG. 3A. The first cross-sectional view in FIG. 3A is perpendicular to the second cross-sectional view in FIG. 3B. With collective reference to FIGS. 3A and 3B, the mechanism 12A further comprises a longitudinal axis 44 and a pressing device 46. The pressing device 46 of this example comprises two arms 48 and two hinges 50. The pressing device 46 is centered with respect to the longitudinal axis 44. Each hinge 50 is perpendicular to, and offset from, the longitudinal axis 44. In this example, each hinge 50 is a living hinge.

Each arm 48 can rotate about a respective hinge 50. In this way, the pressing device 46 can move from a starting position 52, as shown in FIGS. 3A and 3B, to a pressing position.

The pressing device 46 further comprises gripping fingers 56. The gripping fingers 56 constitute one example of a gripping structure according to the present disclosure. The gripping fingers 56 are angled slightly in the distal direction 42. The gripping fingers 56 grip the tab 30 such that the syringe 14 is prevented from moving out from the mechanism 12a in the proximal direction 40. By means of the gripping fingers 56 engaging the tab 30, a firm holding of the syringe 14 by the mechanism 12a is provided.

In FIGS. 3A and 3B, the needle 58 of the syringe 14 can be seen. The needle 58 is concentric with the longitudinal axis 44.

Again, the needle cover 18a constitutes the actuating element 22a in this example. Thus, when reference is made to the needle cover 18a, such reference equally applies to the actuating element 22a in this example.

The needle cover 18a is movable from a ready state 60, as illustrated in FIGS. 3A and 3B, to a dosing state. This movement can be accomplished manually. The needle cover 18a of this example is rigid and centered with respect to the longitudinal axis 44.

The needle cover 18a is also movable from a covered state 62, as illustrated in FIGS. 3A and 3B, to an exposed state. Since the needle cover 18a constitutes the actuating element 22a in this example, the ready state 60 and the covered state 62 are the same state, and the dosing state and the exposed state are the same state. In the covered state 62, the needle cover 18a covers the needle 58. The mechanism 12a of this example further comprise a cover spring 64. The cover spring 64 is one example of a cover force device according to the present disclosure. The cover spring 64 is here a compression coil spring acting between the housing 16 and the needle cover 18a. The cover spring 64 is concentric with the longitudinal axis 44. Moreover, the cover spring 64 surrounds the needle 58 and the needle shield 36.

The mechanism 12a of this example further comprises a movable member 66. In this example, the movable member 66 has a generally cylindrical shape. The movable member 66 is centered with respect to the longitudinal axis 44. The movable member 66 comprises an engageable structure 68. The engageable structure 68 is here exemplified as a proximal end of the movable member 66.

The mechanism 12a of this example further comprises a pressing spring 70. The pressing spring 70 is one example of a pressing force device according to the present disclosure. The pressing spring 70 is here a compression coil spring acting between the housing 16 and the movable member 66. The pressing spring 70 forces the movable member 66 in the proximal direction 40.

The pressing spring 70 is concentric with the longitudinal axis 44. Moreover, the pressing spring 70 is here provided at a distal end of the mechanism 12a and surrounds the tab 30.

The mechanism 12a of this example further comprises a locking arrangement 72. The locking arrangement 72 of this example comprises two legs 74 provided on the needle cover 18a. Each leg 74 is inclined towards the longitudinal axis 44 and in the distal direction 42. As shown in FIG. 3A, the legs 74 engage the engageable structure 68 of the movable member 66. The movable member 66 is thereby prevented from moving in the proximal direction 40.

As long as the movable member 66 is in the position shown in FIGS. 3A and 3B, the pressing device 46 remains in the starting position 52. Movement of the movable member 66 in the proximal direction 40 causes the pressing device 46 to move from the starting position 52 to the pressing position, as described below. Thus, by means of the locking arrangement 72, the needle cover 18a in the ready state 60 prevents movement of the pressing device 46 to the pressing position, here by preventing movement of the movable member 66.

As shown in FIG. 3B, a first part of the movable member 66 is aligned with the distal window 24. A medicament reservoir of the syringe 14 is visible through the proximal window 26. A user can confirm that the syringe 14 comprises medicament by looking into the proximal window 26.

The cap 20 comprises an insert 76, here made of metal. A user can remove the cap 20 by rotating the cap 20 in the first direction 28 about the longitudinal axis 44. The first direction 28 is a counterclockwise rotation as seen in the distal direction 42. When the cap 20 is rotated in the first direction 28, the insert 76 grips the needle shield 36 such that the needle shield 36 rotates together with the cap 20. In this way, the cap 20 can be removed from the medicament delivery device 10a together with the needle shield 36.

Should the cap 20 be rotated in a second direction opposite to the first direction 28 (i.e. in a clockwise rotation as seen in the distal direction 42), the insert 76 slides over the needle shield 36 such that the cap 20 rotates relative to the needle shield 36 (which is then stationary). The insert 76 thus only grips the needle shield 36 when the cap 20 is rotated in the first direction 28. By means of the insert 76, the cap 20 functions as a freewheel device.

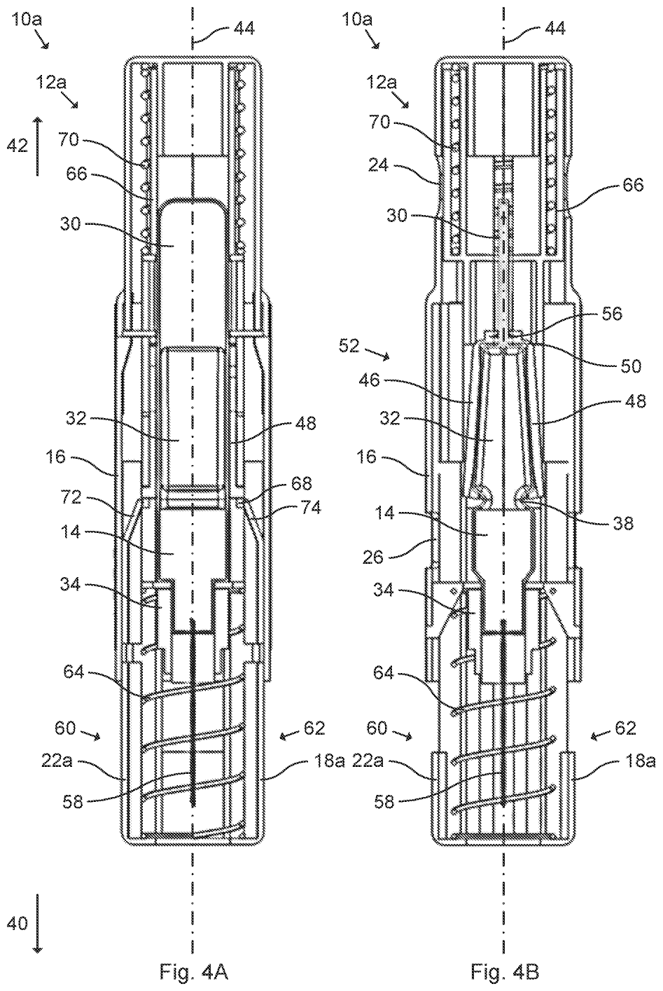

FIG. 4A schematically represents a first cross-sectional side view of the medicament delivery device 10a after removal of the cap 20, and FIG. 4B schematically represents a second cross-sectional side view of the medicament delivery device 10a in FIG. 4A. When the needle shield 36 is rotated by rotation of the cap 20, the needle 58 also moves in the distal direction 42 to pierce into the medicament reservoir. Rotation of the cap 20 and the needle shield 36 causes these parts to eventually disengage for removal.

The cover spring 64 forces the needle cover 18a in the proximal direction 40. The cover spring 64 thereby forces the needle cover 18a to the covered state 62. In the covered state 62 the needle cover 18a protects against accidental sticks by the needle 58.

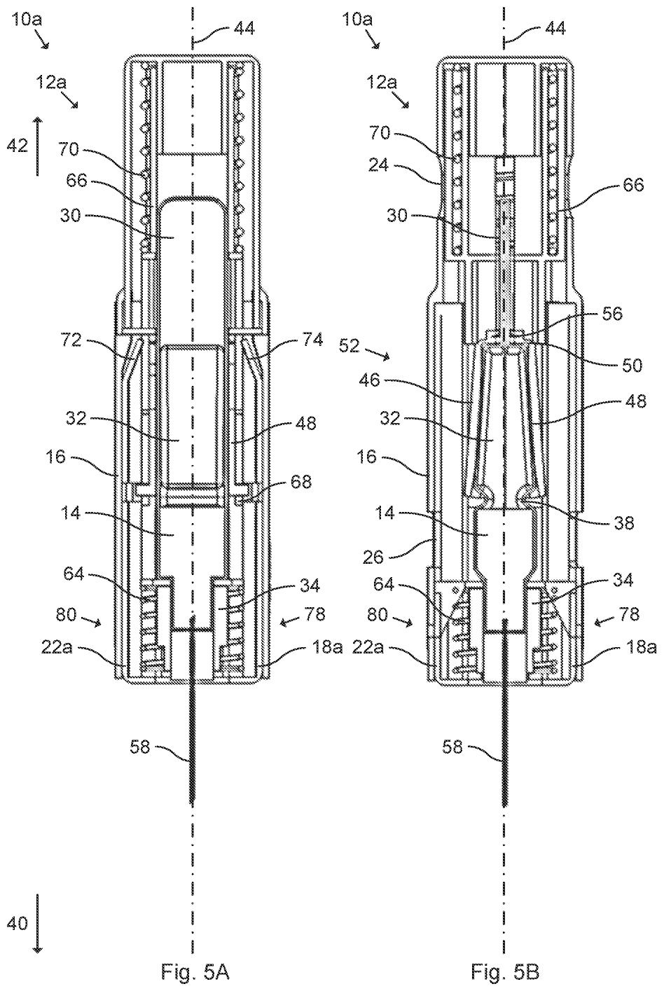

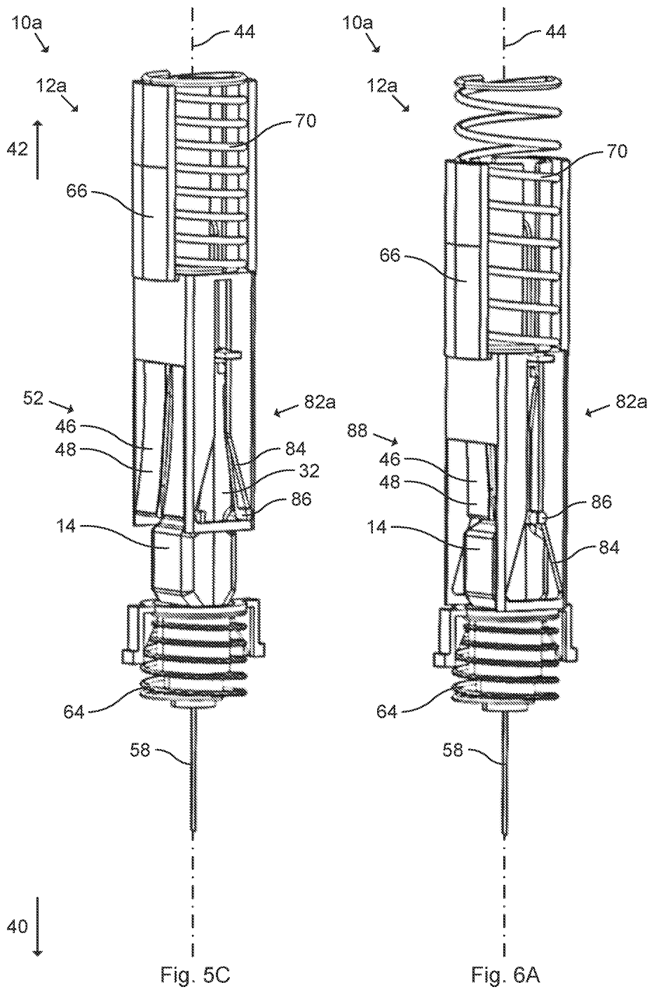

FIG. 5A schematically represents a first cross-sectional side view of the medicament delivery device 10a, and FIG. 5B schematically represents a second cross-sectional side view of the medicament delivery device 10a in FIG. 5A. With collective reference to FIGS. 5A and 5B, the needle cover 18a has moved in the distal direction 42 relative to the housing 16 from the covered state 62 to the exposed state 78. Since the needle cover 18a of this example also constitutes an actuating element 22a, the movement of the needle cover 18a from the covered state 62 to the exposed state 78 also constitutes a movement from the ready state 60 to the dosing state 80.

The needle cover 18a moves from the covered state 62 to the exposed state 78 against the force of the cover spring 64. The cover spring 64 thereby becomes compressed, or becomes more compressed, in comparison with FIGS. 4A and 4B. In the exposed state 78, the needle cover 18a does not cover the needle 58.

The needle cover 18a may transition from the covered state 62 to the exposed state 78 when a user grabs the medicament delivery device 10a and presses the needle cover 18a against an injection site. This will cause the needle cover 18a to move in the distal direction 42 and the needle 58 to pierce the injection site.

The pressing of the needle cover 18a in the distal direction 42 causes the legs 74 to snap over the engageable structure 68, as shown in FIG. 5A. As a consequence, movement of the movable member 66 in the proximal direction 40 is no longer blocked by the needle cover 18a. The force in the pressing spring 70 is thereby released when the needle cover 18a adopts the dosing state 80.

FIG. 5C schematically represents a partial perspective view of the medicament delivery device 10a in FIGS. 5A and 5B. As shown in FIG. 5C, the mechanism 12a further comprises a force transmission arrangement 82a. The force transmission arrangement 82a of this example comprises the movable member 66. The force transmission arrangement 82a of this example further comprises cam profiles 84 and cam followers 86 arranged to follow the cam profiles 84. The cam followers 86 are here provided in the pressing device 46 and the cam profiles 84 are here provided on the movable member 66. However, this configuration may be switched.

Two cam followers 86 are provided on one of the arm 48 and two cam followers 86 are provided on the other arm 48. The cam followers 86 are here aligned with the recesses 38 (FIG. 3B). The cam followers 86 are here exemplified as pins.

The pressing spring 70 now forces the movable member 66 to move in the proximal direction 40. The causes the cam followers 86 to travel along the respective cam profiles 84. As a consequence, the arms 48 are forced towards each other and the injection starts. That is, the arms 48 press the bubble 32 such that medicament is expelled through the needle 58. The force transmission arrangement 82a is thereby configured to transmit a movement of the needle cover 18a from the covered state 62 to the exposed state 78 to a movement of the pressing device 46 from the starting position 52 to the pressing position via release of the movable member 66.

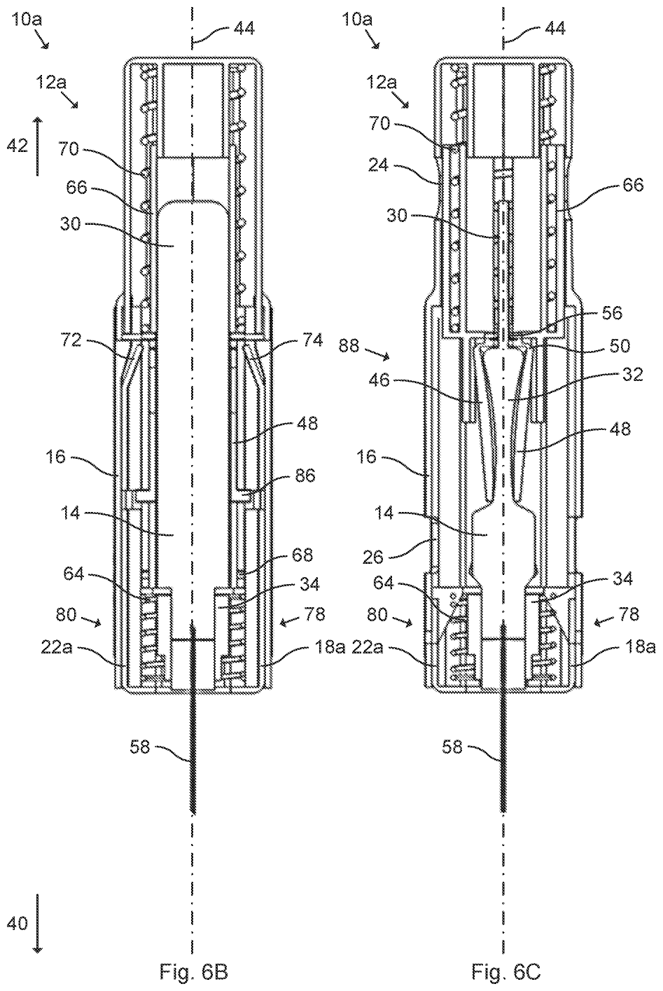

FIG. 6A schematically represents a partial perspective view of the medicament delivery device 10a, FIG. 6B schematically represents a first cross-sectional side view of the medicament delivery device 10a in FIG. 6A, and FIG. 6C schematically represents a second cross-sectional side view of the medicament delivery device 10a in FIGS. 6A and 6B. In FIGS. 6A-6C, the arms 48 have been pressed together inwardly such that the pressing device 46 adopts the pressing position 88. The arms 48 move relative to the housing 16. The needle cover 18a remains in the exposed state 78 until the arms 48 have performed full motion. The pressing spring 70 thus pushes the movable member 66 which in turn forces the arms 48 together by means of the cam profiles 84 and the cam followers 86. The pressing spring 70 is thereby arranged to force the pressing device 46 to the pressing position 88.

Since the movable member 66 has moved in the proximal direction 40, a second part of the movable member 66 is now aligned with the distal window 24. The second part may have a color different from the first part. The alignment of the second part with the distal window 24 confirms medicament delivery to the user. In addition, the user can see through the proximal window 26 that the medicament has been delivered from the syringe 14.

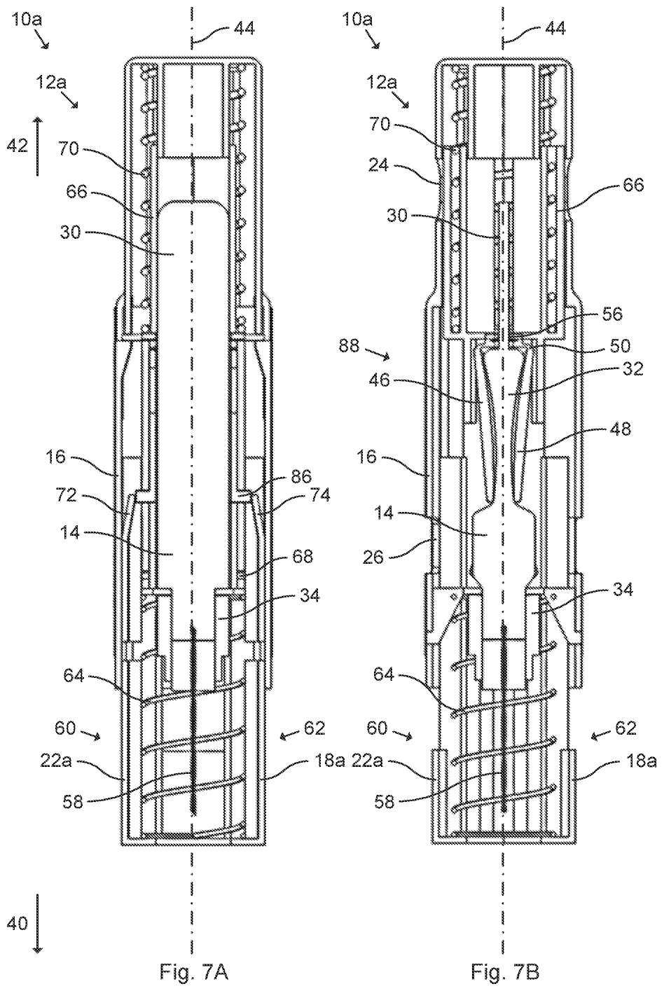

FIG. 7A schematically represents a first cross-sectional side view of the medicament delivery device 10a, and FIG. 7B schematically represents a second cross-sectional side view of the medicament delivery device 10a in FIG. 7A. With collective reference to FIGS. 7A and 7B, the medicament delivery device 10a has now been removed from the injection site. The cover spring 64 thereby forces the needle cover 18a to transition from the exposed state 78 back to the covered state 62 by movement in the proximal direction 40.

The legs 74 of the needle cover 18a snaps over the cam followers 86 when the needle cover 18a moves in the proximal direction 40. Thus, when the pressing device 46 has adopted the pressing position 88, the pressing device 46 prevents the needle cover 18a from being moved from the covered state 62 to the exposed state 78. The locking arrangement 72 thereby locks the needle cover 18a in the covered state 62 after having transitioned from the exposed state 78 to the covered state 62. The entire medicament delivery device 10a, including the previously removed cap 20 and needle shield 36, can now be disposed.

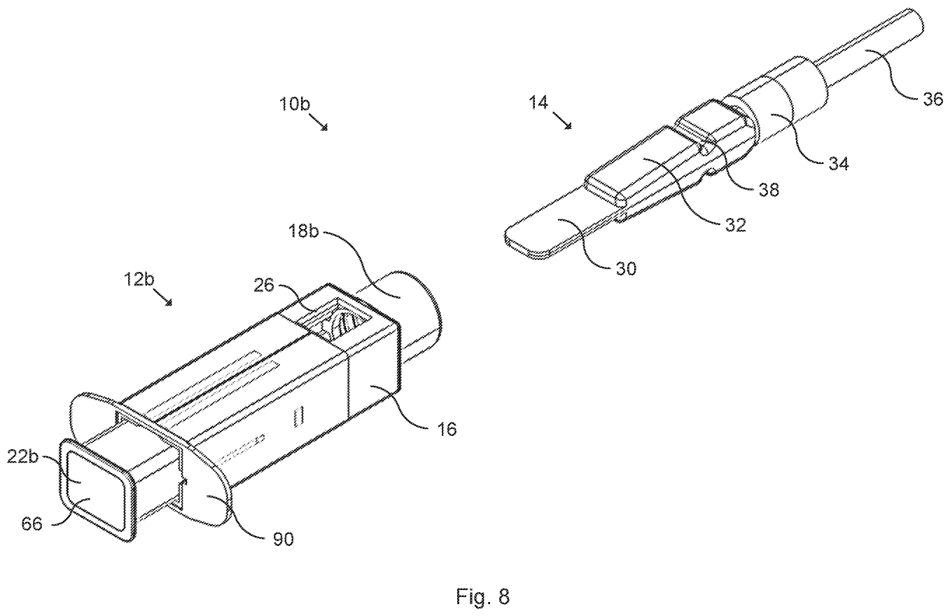

FIG. 8 schematically represents a perspective and partially exploded view of a medicament delivery device 10b according to a further example. Mainly differences with respect to the medicament delivery device 10a in FIGS. 1-7B will be described. The medicament delivery device bob comprises an alternative mechanism 12b. The mechanism 12b comprises a needle cover 18b at a proximal end and a button 22b at a distal end. The button 22b is a further example of an actuating element according to the present disclosure. The button 22b is a part of a movable member 66, similar to the movable member 66 in FIGS. 1-7D.

The housing 16 comprises two wings 90. The housing 16 further comprises a single window 26.

In FIG. 8, the syringe 14 is of the same type as in FIGS. 1-7D. The medicament delivery device bob may be delivered in a pre-assembled state to the user. Alternatively, the user (or other caregiver) may connect the syringe 14 to the mechanism 12b at a point-of-care, e.g. by inserting the syringe 14 into the mechanism 12b.

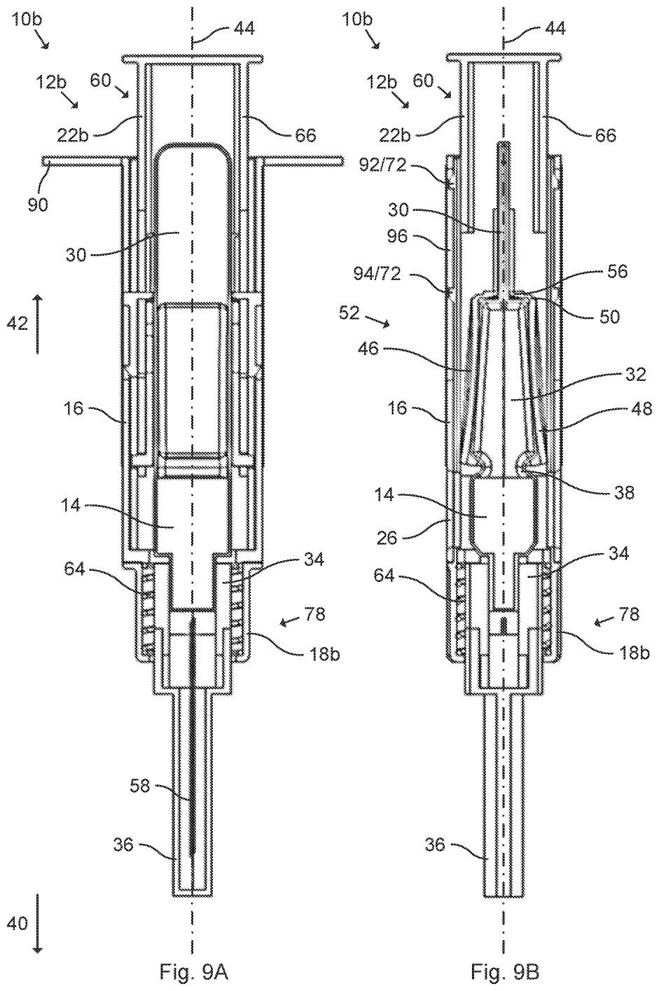

FIG. 9A schematically represents a first cross-sectional side view of the medicament delivery device bob, and FIG. 9B schematically represents a second cross-sectional side view of the medicament delivery device bob in FIG. 9A. The cross-sectional plane in FIG. 9A coincides with the longitudinal axis 44 while the cross-sectional plane in FIG. 9B is offset from the longitudinal axis 44. The mechanism 12b does not comprise any pressing spring. The gripping fingers 56 grip the tab 30 when the syringe 14 is inserted into the mechanism 12b. In FIGS. 9A and 9B, the needle cover 18b is in the exposed state 78.

The needle cover 18b comprises distal protrusions 92 and proximal protrusions 94. As shown in FIG. 9B, the distal protrusions 92 and the proximal protrusions 94 engage the housing 16. The housing 16 comprises slots 96. The distal protrusions 92 and the proximal protrusions 94 engage these slots 96. The needle cover 18b is thereby guided in the housing 16. The distal protrusions 92 and the proximal protrusions 94 constitute a further example of a locking arrangement 72 according to the present disclosure.

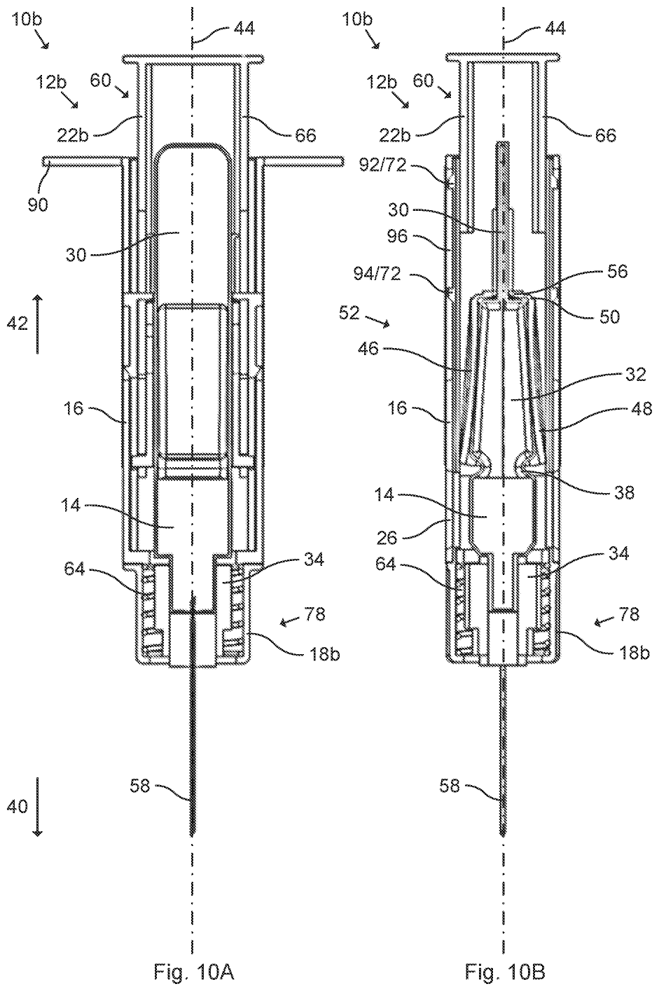

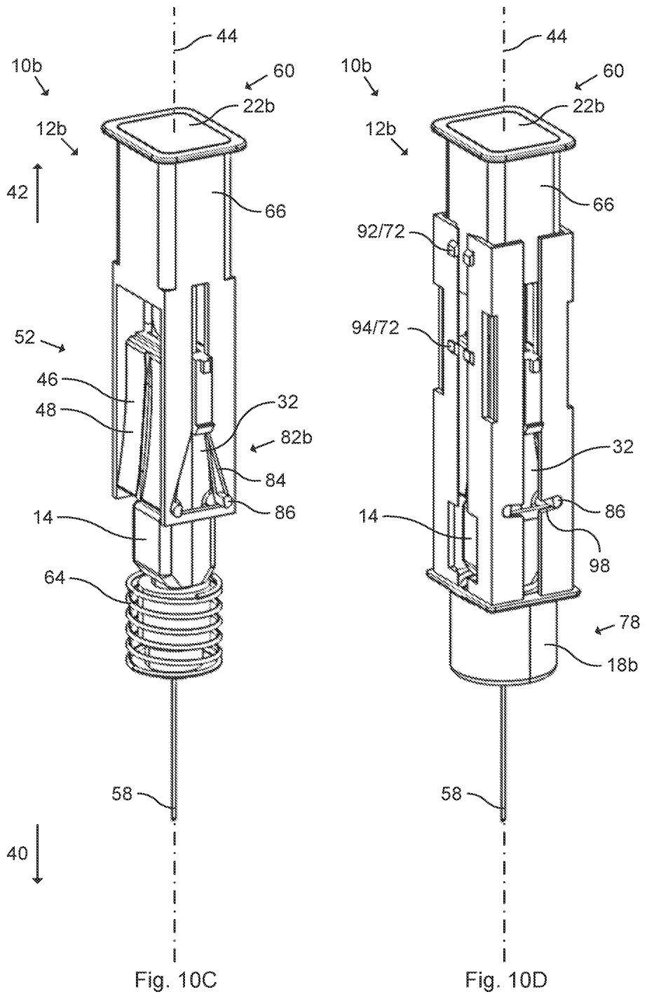

FIG. 10A schematically represents a first cross-sectional side view of the medicament delivery device 10B, and FIG. 10B schematically represents a second cross-sectional side view of the medicament delivery device 10b in FIG. 10A. With collective reference to FIGS. 10A and 10B, the needle shield 36 has been removed. The needle 58 penetrates the medicament reservoir of the syringe 14 when the needle shield 36 is twisted off. Since the needle cover 18b is already in the exposed state 78, the needle 58 is exposed when the needle shield 36 is removed.

FIG. 10C schematically represents a first partial perspective view of the medicament delivery device 10B, and FIG. 10D schematically represents a second partial perspective view of the medicament delivery device 10B. With collective reference to FIGS. 10C and 10D, the mechanism 12b comprises a force transmission arrangement 82b. The force transmission arrangement 82b differs from the force transmission arrangement 82a in that the movable member 66 is driven in the proximal direction 40 by manually pressing the button 22b, instead of by a pressing spring.

The needle cover 18b comprises two tracks 98 (only one track 98 is visible in FIG. 10D). Each track 98 comprises a longitudinal part and two lateral parts, perpendicular to the longitudinal part. Each longitudinal part is parallel with the longitudinal axis 44.

Although the needle cover 18b is forced in the proximal direction 40 by means of the cover spring 64, the engagement of the cam followers 86 in a respective lateral part of the track 98 of the needle cover 18b prevents the needle cover 18b from moving in the proximal direction 40.

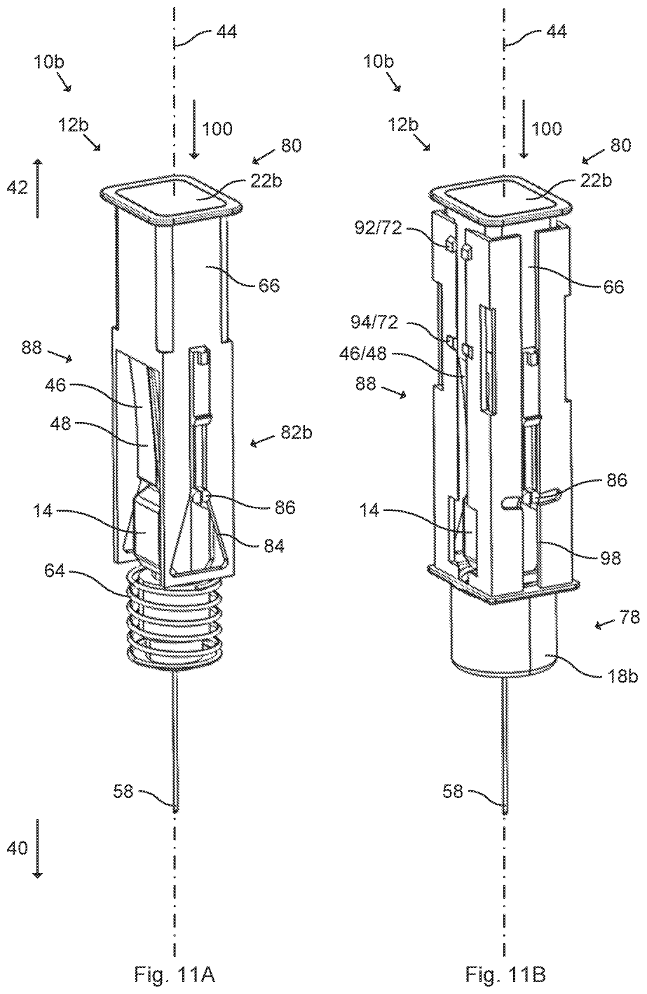

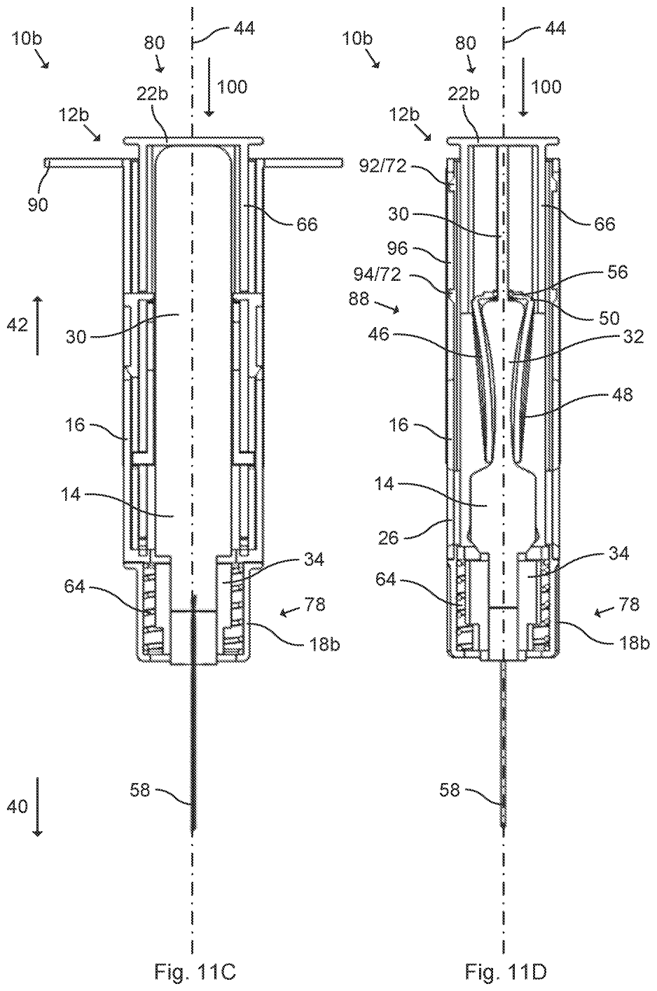

FIG. 11A schematically represents a first partial perspective view of the medicament delivery device 10b, and FIG. 11B schematically represents a second partial perspective view of the medicament delivery device 10b in FIG. 11A. In FIGS. 11A and 11B, the button 22b has been moved from the ready state 60 to the dosing state 80 by means of a manual push, as indicated by arrow 100. The manual push can be accomplished by a user by holding one or more fingers on each wing 90 and pressing the button 22b with the thumb.

The force transmission arrangement 82b transmits the movement of the button 22b from the ready state 60 to the dosing state 80 to a movement of the pressing device 46 from the starting position 52 to the pressing position 88 by means of the cam followers 86 and the cam profiles 84, in the same way as described for the mechanism 12a. When the pressing device 46 adopts the pressing position 88, the arms 48 have been brought together to expel the medicament through the needle 58. The cam followers 86 are now brought close together.

In the pressing position 88, the cam followers 86 no longer engage the lateral part of the track 98. Instead, the cam followers 86 are now free to move along the longitudinal parts of the track 98. The needle cover 18b is thereby free to move in the proximal direction 40 when the user lifts the medicament delivery device 10b away from the injection site.

FIG. 11C schematically represents a first cross-sectional side view of the medicament delivery device 10b in FIGS. 11A and 11B, and FIG. 11D schematically represents a second cross-sectional side view of the medicament delivery device 10b in FIGS. 11A and 11B. With reference to FIG. 11D, the user can see through the window 26 that the medicament reservoir of the syringe 14 is empty.

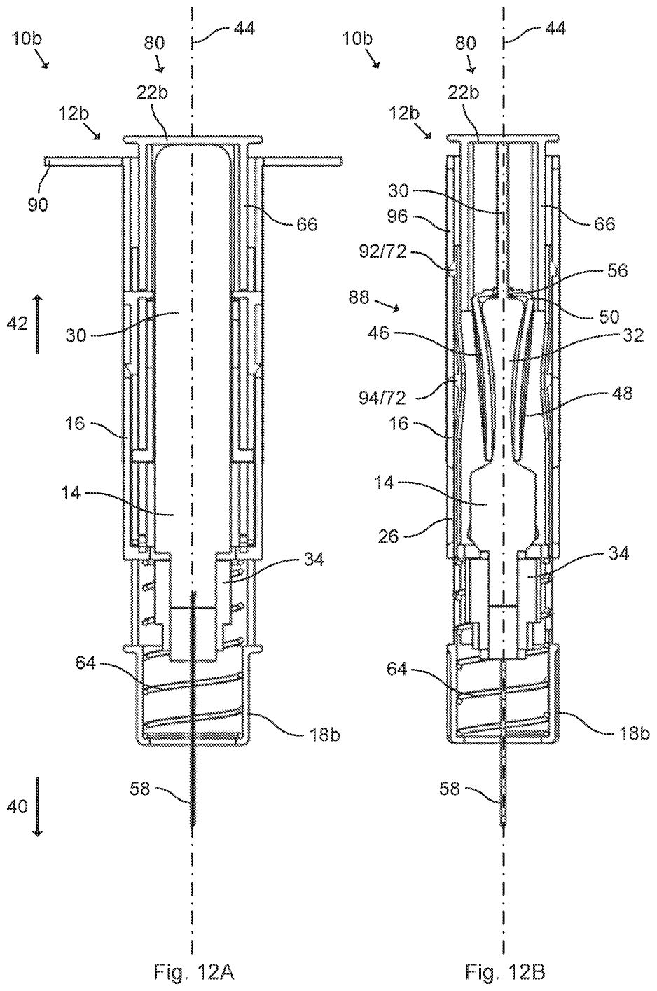

FIG. 12A schematically represents a first cross-sectional side view of the medicament delivery device 10b, and FIG. 12B schematically represents a second cross-sectional side view of the medicament delivery device 10b in FIG. 12A. In FIGS. 12A and 12B, the needle cover 18b moves in the proximal direction 40 by the force of the cover spring 64 as the medicament delivery device 10b is lifted away from the injection site. During this movement, the cam followers 86 move along the longitudinal parts of the track 98. As shown in FIG. 12B, the needle cover 18b flexes laterally inwards when the proximal protrusions 94 move relative to the housing 16 and leave the slots 96.

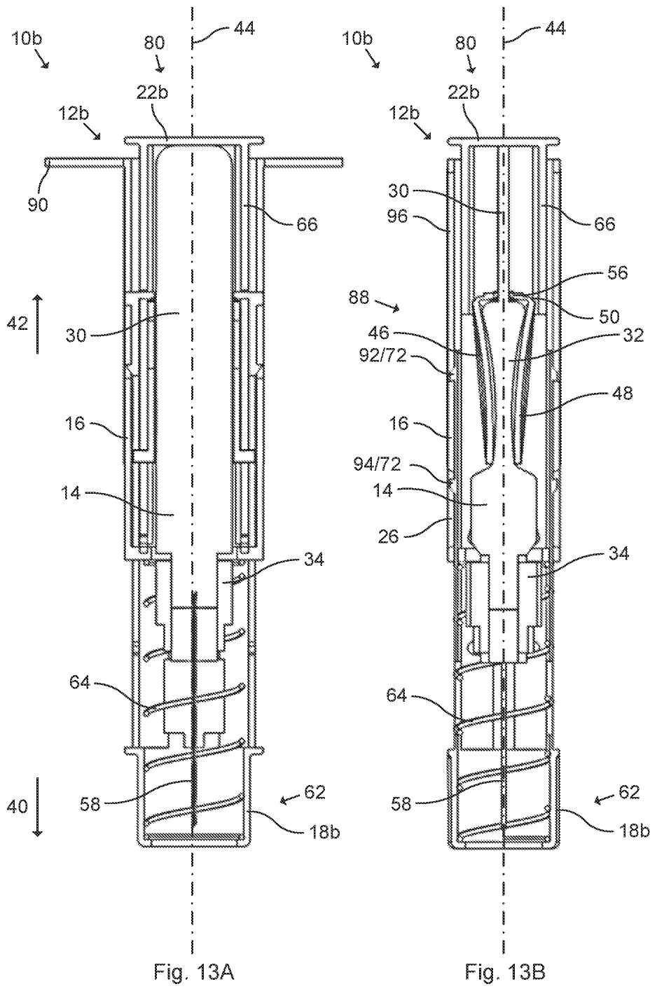

FIG. 13A schematically represents a first cross-sectional side view of the medicament delivery device 10b, and FIG. 13B schematically represents a second cross-sectional side view of the medicament delivery device 10b in FIG. 13A. In FIGS. 13A and 13B, the needle cover 18b has moved in the proximal direction 40 to the covered state 62. In the covered state 62, the proximal protrusions 94 have engaged the window 26 and the distal protrusions 92 have engaged a proximal end of the slots 96. The proximal protrusions 94 thereby prevent movement of the needle cover 18b in the distal direction 42, and the distal protrusions 92 thereby prevent movement of the needle cover 18b in the proximal direction 4o. The needle cover 18b is therefore locked in the covered state 62. The entire medicament delivery device 10b can then be disposed.

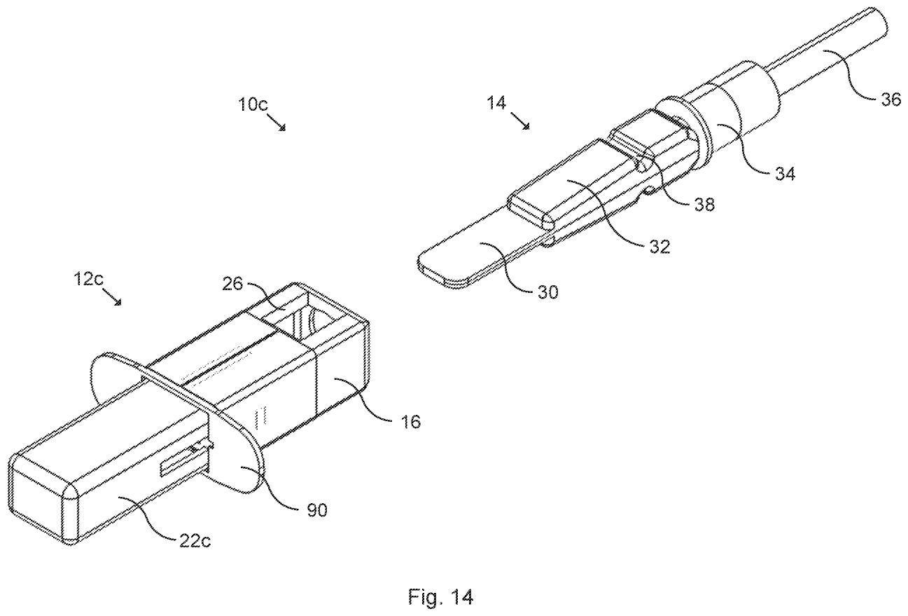

FIG. 14 schematically represents a perspective and partially exploded view of a medicament delivery device 10c according to a further example. Mainly differences with respect to the medicament delivery device 10b will be described. The medicament delivery device 10c comprises a mechanism 12C. The mechanism 12C comprises a button 22C. The button 22C is a further example of an actuating element according to the present disclosure.

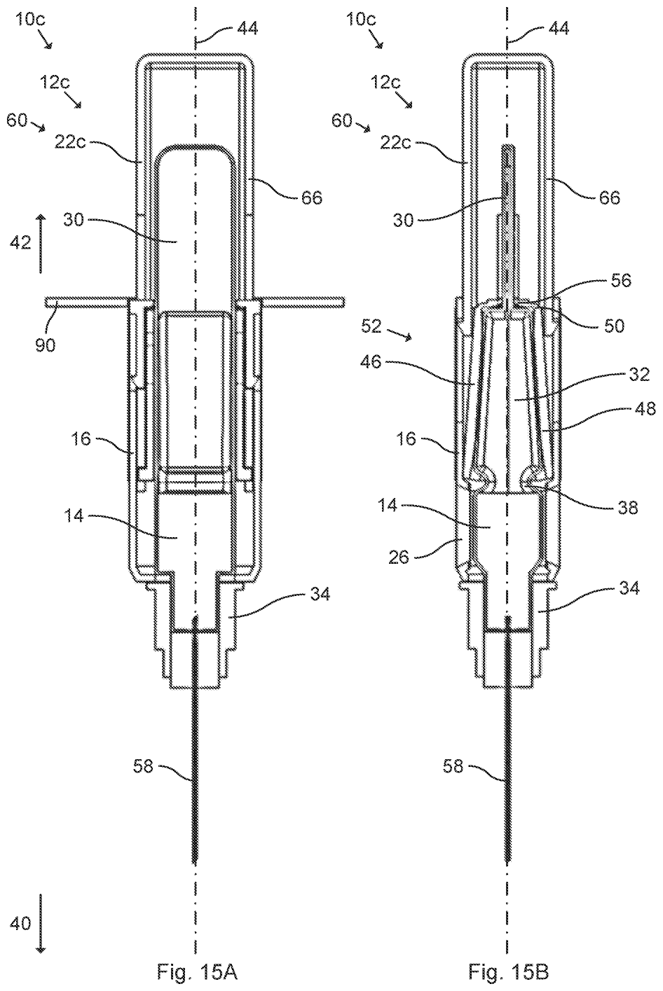

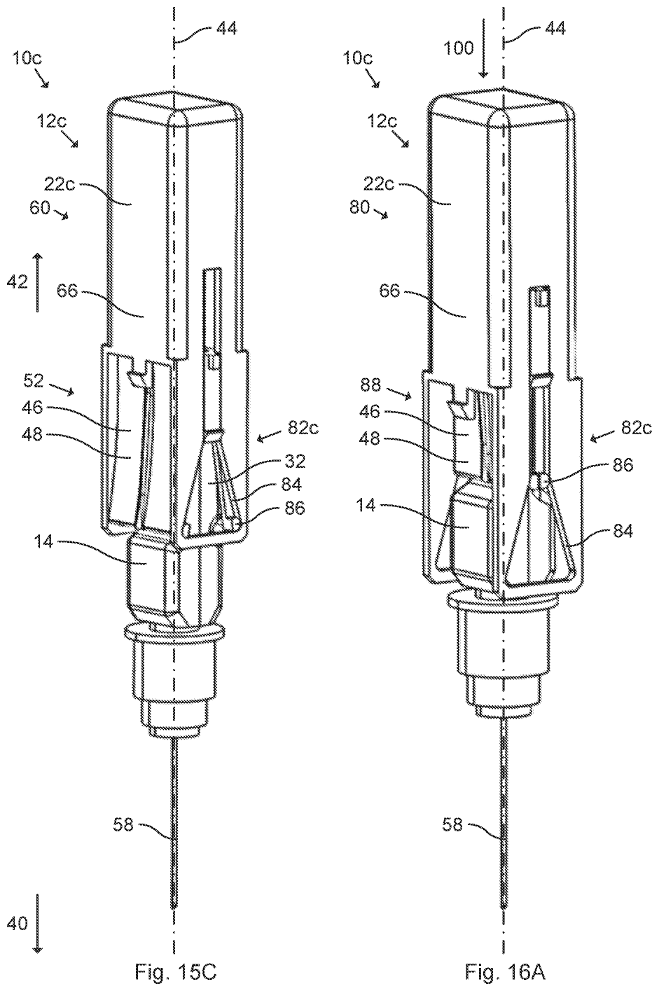

FIG. 15A schematically represents a first cross-sectional side view of the medicament delivery device 10c, FIG. 15B schematically represents a second cross-sectional side view of the medicament delivery device 10c, and FIG. 15C schematically represents a partial perspective view of the medicament delivery device 10c in FIGS. 15A and 15B. The mechanism 12C does not comprise any cover spring. The button 22C may be transparent. In this way, information on the tab 30 can be read in an assembled state of the medicament delivery device 10c. The mechanism 12C comprises a force transmission arrangement 82c of the same type as the force transmission arrangement 82b.

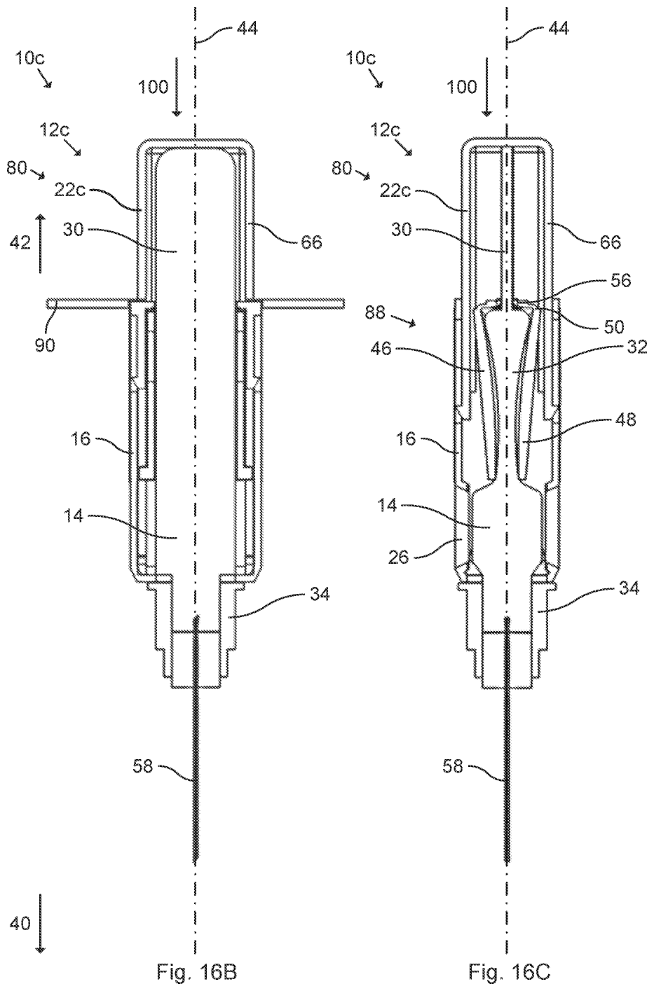

Once the needle shield 36 has been removed, as shown in FIGS. 15A-15C, the user can pierce the needle 58 into an injection site and press the button 22C. FIG. 16A schematically represents a partial perspective view of the medicament delivery device 10c, FIG. 16B schematically represents a first cross-sectional side view of the medicament delivery device 10c in FIG. 16A, and FIG. 16C schematically represents a second cross-sectional side view of the medicament delivery device 10c in FIGS. 16A and 16B. In FIGS. 16A-16C, the pressing device 46 has moved from the starting position 52 to the pressing position 88 by movement of the button 22C from the ready state 60 to the dosing state 80. The used medicament delivery device 10c in FIGS. 16A-16C is shorter than the unused medicament delivery device 10c in FIGS. 15A-15C.

While the present disclosure has been described with reference to exemplary embodiments, it will be appreciated that the present invention is not limited to what has been described above. For example, it will be appreciated that the dimensions of the parts may be varied as needed. Accordingly, it is intended that the present invention may be limited only by the scope of the claims appended hereto.

1-15. (canceled)

16: A mechanism for receiving an injector having a needle and a compressible body for driving expulsion of medicament through the needle, the mechanism comprising:
an actuating element configured to transition from a ready state to a dosing state by means of manual manipulation;
a pressing device arranged to move from a starting position to a pressing position to thereby press the compressible body when the injector is received by the mechanism; and
a force transmission arrangement configured to transmit a transition of the actuating element from the ready state to the dosing state to a movement of the pressing device from the starting position to the pressing position.

17: The mechanism according to claim 16, wherein the force transmission arrangement comprises a cam profile and a cam follower arranged to follow the cam profile and wherein the cam profile or the cam follower is provided on the pressing device.

18: The mechanism according to claim 16, wherein the force transmission arrangement comprises a movable member, and wherein the cam profile or the cam follower is provided on the movable member.

19: The mechanism according to claim 16, further comprising a needle cover configured to transition from an exposed state, where the needle cover does not cover the needle, to a covered state, where the needle cover covers the needle, and a cover force device arranged to force the needle cover towards the covered state.

20: The mechanism according to claim 21, wherein the needle cover is prevented by the pressing device from transitioning to the covered state when the pressing device adopts the starting position, and allowed by the pressing device to transition to the covered state when the pressing device adopts the pressing position, and wherein the actuating element comprises the needle cover.

21: The mechanism according to claim 16, wherein the pressing device comprises two arms arranged to move towards each other when the pressing device moves from the starting position to the pressing position.

22: The mechanism according to claim 16, further comprising a locking arrangement arranged to lock the needle cover in the covered state after having transitioned from the exposed state to the covered state.

23: The mechanism according to claim 16, wherein the injector comprises a tab on a distal side of the compressible body, and wherein the mechanism comprises a gripping structure arranged to grip the tab when the injector is received by the mechanism, and wherein the gripping structure is provided in the pressing device.

24: The mechanism according to claim 16, wherein the injector further comprises a needle shield arranged to be unidirectionally unscrewed to expose the needle, and wherein the mechanism further comprises a cap arranged to rotationally engage the needle shield by rotation of the cap.

25: The mechanism according to claim 16, further comprising a pressing force device arranged to force the pressing device to the pressing position.

26: The mechanism according to claim 30, wherein the mechanism is configured such that a force from the pressing force device is released when the actuating element adopts the dosing state.

27: The mechanism according to claim 30, wherein the pressing force device comprises a spring.

28: The mechanism according to claim 30, when depending on claim 4, wherein the pressing force device is arranged to force the movable member.

29: The mechanism according to claim 16, wherein the actuating element is configured to prevent movement of the pressing device to the pressing position when the actuating element adopts the ready state.

30: A medicament delivery device comprising the injector and a mechanism according to claim 16.

31: An assembly for expelling medicament through a needle, the assembly comprises:
an injector comprising a compressible body that is attached to the needle;
an actuating element that transitions from a ready state to a dosing state by user manual manipulation;
a pressing device that moves from a starting position to a pressing position to exert pressure on the compressible body when the injector is received into the assembly; and
a cam and cam follower combination associated with the pressing device that transmits a transition of the actuating element from the ready state to the dosing state to a movement of the pressing device from the starting position to the pressing position.

32: The assembly of claim 31 further comprising a needle cover operatively associated with a cover force device that biases the needle cover in a proximal direction.

33: The assembly of claim 32, wherein the pressing device operative interacts with the needle cover to prevent the needle cover from moving to a covered state when the pressing device is in the starting position.

34: The assembly of claim 31, wherein the pressing device comprises two arms that move towards each other during transition to the pressing position.

35: The assembly of claim 31 further comprising a tab on the compressible body that engages with a gripping structure on the pressing structure when the injector is positioned in the assembly.
